(19) United States
(12) Patent Application Publication
(10) Pub. No.: US 2016/0015992 A1
(43) Pub. Date: Jan. 21, 2016

(54) ICD/VAD DEFIBRILLATOR ELECTROMAGNET

(71) Applicant: Garrick Whitlock, Pawtucket, RI (US)

(72) Inventor: Garrick Whitlock, Pawtucket, RI (US)

(21) Appl. No.: 14/801,517

(22) Filed: Jul. 16, 2015

Related U.S. Application Data
(60) Provisional application No. 62/026,488, filed on Jul. 18, 2014.

(51) Int. Cl. A61N 1/39

(52) U.S. Cl. CPC A61N 1/3931; A61N 1/3956

(57) ABSTRACT

The ICD/VAD defibrillator electromagnet is used with patients who possess either an ICD or a VAD. The electromagnet has a slim, low-profile design and an adhesive pad that allows for proper positioning to patient's chest, a decrease risk for pressure related wounds, and also permits the positioning of the patient in any of multiple planes while in surgery/X-ray without worry about displacement or shifting of the electromagnet. The electromagnet provides protection and safety for patients undergoing surgery involving electrocaurtery as well as, helping patients in X-ray/CT/MRI procedures that produce high magnetic fields that interfere with ICD/VAD proper function.

### RELATED APPLICATION

This application claims the benefit of U.S. Provisional Application Ser. No. 62/026,488 entitled “ICD/VAD Defibrillator Electromagnet” that was filed on Jul. 18, 2014 which is incorporated herein by this reference.

### BACKGROUND OF THE INVENTION

1. External Medical Device

The present invention relates to medically implanted devices (MID) such as Implanted Cardiovertor Defibrillators (ICDs) and/or Ventricular Assist Devices (VADs) and methods for protecting the MID from either Conductive or Radiated Electromagnetic Interference (EMI). More specifically, the present invention relates to the ability to place the ICD/VAD in a triggered or asynchronous mode. ICD/VADs now can have the arrhythmia detection suspended intermittently at a medical provider's discretion or in concert with external generator activation by a surgeon before, during, and after EMI producing activities are suspected to accrue. The present invention also relates to methods that protect the MID from electromagnetic interference (EMI) that may alter the performance of implanted cardiac devices and provides strategies to minimize negative effects on patient hemodynamic status.

2. The Relevant Technology

Patients with MIDs may be subjected to medical procedures in which EMI producing equipment (e.g., electrosurgery, cautery or RF ablation) is used. Some types of interference may not be filtered out by MID sensing and may be erroneously interpreted as a rapid heart rate. If persistent, this interference could cause the tachyarrhythmia detection criterion to be met and a tacharrhythmia therapy to be inappropriately delivered. Accordingly, there are instances when it is desirable to temporarily suspend the tachyarrhythmia detection capabilities of an MID.

Most pacemakers and ICDs have built-in magnetic reed switches that are designed to switch “ON” or “OFF” the tachyarrhythmia detection circuitry in response to magnets. Some newer devices are equipped with alternative technologies like giant magnetosensitive resistors (GMRs), Hall-effect sensors, or telemetry coils that also respond to magnets.

Clinical magnets, made of ferrous alloy, are known in the industry and come in various shapes (ring or doughnut, horseshoe, and rectangle or bar) and are design to overlay the ICD/VAD implantation site. Such clinical magnets are permanent magnets that cannot be turned off and on. By properly placing such clinical magnets proximate the MID, the operation of the MID can be temporarily suspended until the clinical magnet is removed from its proper placement. Unfortunately, such clinical magnets move inadvertently during medical procedures prematurely allowing the MID to operate and/or the clinical magnets can cause wounds to the patient by tight adherence or undesired pressure at the clinical magnet site.

Several clinical magnets are known in the art. These clinical magnets require proper placement as per manufacturer (white papers) recommendations. Ring/doughnut magnets and bar magnets, the St. Jude Telemetry Wand magnet with a removable magnet, and the Medtronic Smart Magnet™ are known and have been used to turn off implantable cardioverter defibrillators.

One type of clinical magnet is produced by Medtronic (Minneapolis, Minn., USA) and is known as the Smart Magnet™. The Medtronic Smart Magnet™ has a light indicator to guide appropriate placement and is a permanent magnet produced with an external, rigid injection-molded enclosure to withstand robust handling. Because the Smart Magnet™ is a permanent magnet, its magnetic field cannot be turned off and on or varied in strength. Rather, its magnetic field has a given strength and is always on. To remove its effect on an MID, it must be removed from positioning proximate the MID. Also, the Smart Magnet™ is sold without means provided to adhere it to a patient. Hence, external means of adherence such as adhesive, tape, or elastic wrap must be used to secure the magnet in the vicinity needed to properly suspend operation of the MID. Likewise, the external means of adherence such as adhesive, tape, or elastic wrap must be removed to allow the magnet to be removed from the vicinity of the MID to allow operation of the MID to recommence. If the Smart Magnet™ needs to be reintroduced during the medical procedure, it must be repositioned properly and re-adhered to make certain that it has the desired effect on the MID. When placed properly, the Smart Magnet™ results in the expected response from MIDs; however, the indicator illuminates only when used with Medtronic MIDs.

Another type of clinical magnet is the St. Jude Telemetry Wand magnet which is also available for use with ICD/VADs. Again, the St. Jude Telemetry Wand magnet is a permanent magnet that cannot be turned off and on, although the magnet may be removed from the wand.

The magnetic field effect of each of these clinical magnets is directly proportional to the strength of the magnet and inversely proportional to the distance of the magnet from the ICD/VAD. These available clinical magnets usually have a strength of >90 Gauss. With each of these clinical magnets, temporary adhesion of the magnet to the skin is a challenge due to difficult patient positioning (lateral/prone), body size, or the length of procedure.

It will also be appreciated that when using presently available clinical magnets, the current magnet systems are difficult to position correctly on a prone or lateral patient and on morbidly obese patients. The current magnets also increase the chance of pressure wounds (skin breakdown) due either to increased duration of procedure or external pressure forces from positioning devices. Consequently, patients frequently experience discomfort due to the improper positioning of the magnets or due to positioning failure and/or dislodging during a procedure. Patients also experience pressure wounds caused by the size and rigidity of the clinical magnets.

It would be an advancement in the art for a magnet to alleviate such problems while providing dependable suspension of the MID's operation.

### BRIEF SUMMARY OF THE INVENTION

The present disclosure has been developed in response to the current clinical magnets available on the market, and in particular, in response to the problems of improper positioning of clinical magnets, movement of the clinical magnet on patients once positioned, draped, anesthetized, or placed in X ray/CT/MRI; as well as, the pressure wound potential from clinical magnets and external forces from positioning a patient prone/lateral or for prolonged surgical duration. These are needs in the clinical magnets that have not yet been fully resolved by magnets currently available in the marketplace.

The exemplary electromagnet of the present disclosure is made to be placed directly over the patients' ICD/VAD, enabling proper temporary adhesion of the electromagnet to the skin due to difficult patient positioning (lateral/prone), body size, or length of procedure and provides protection from use of either a conductive or radiated EMI source. The exemplary electromagnet comprises a thin wafer wrapped in conductive wire (such as copper or other conductive metallic wire). The thin wafer may have any of numerous shapes so long as it maintains a low profile when wound with the conductive wire. Additionally, the thin wafer may be made of any material that becomes electromagnetic when electrical current is introduced to the conductive wire.

Positive and negative poles are established so that the wire carries an electrical current to produce the electromagnet. The electromagnet is disposed inside an insulated gel adhesive pad with an electrical cord attached to a controller which is attached to a suitable power source such as an external battery pack or an AC outlet. Of course, electromagnets of various strengths may have differing numbers of wrappings (or windings) around the thin wafer. Also, a standard electromagnet may have a predetermined number of wrappings of conductive wire about the thin wafer, and still have adjustable magnetic strength by adjusting the amount of electrical current sent through the wrappings of conductive wire.

The controller may have an LED light indicator that lights when the electromagnet is activated. The power source may be any suitable power source such as, for example, the external battery pack mentioned, an AC power source and inverter, or an external electrocautery generator source (e.g., Bovie generator, APC/Erby Generator).

If the external electrocautery generator is the source, a time relay resistor with a short delay (5-10 seconds, for example) may be provided so that the electromagnet is activated shortly before the electrocautery device and remains active until shortly after the electrocautery device is deactivated. This resistor, once activated by the external electrocautery generator, triggers the ICD/VAD, placing it in an asynchronous mode before the EMI source is activated and used, and maintains that asynchronous mode until after the EMI source is deactivated. Again, the controller may have an LED light indicator that illuminates when the electromagnet is active. This allows the external electrocautery generator to power the electromagnet and to serve as a grounding source as well.

The defibrillator electromagnet structure of the exemplary embodiments of the present disclosure may have the same or similar ferrous alloy design, but has a substantially slimmer profile design, weight less, and has an adhesive pad to help with better positioning and adhesion of electromagnet compared to magnets currently on the market for use with MIDs. The ability to trigger the asynchronous mode through an external battery pack, external generator, or other power source allows for more effective management of patients hemodynamic status and allows the ICD/VAD to function at optimal settings to benefit the patient.

The exemplary defibrillator electromagnets of the present disclosure are activating/deactivating non-invasive ICD/VAD electromagnets having a low profile and may be disposed in a non-conductive adhesive gel pad to aide with correct positioning over the ICD/VAD. The slim profile also decreases the risk of pressure wounds due to prone/lateral positioning or prolonged procedures. The exemplary electromagnets have the ability to suspend arrhythmia detection intermittently at an operator's (i.e., medical provider) discretion or in concert with external generator activation by an operator surgeon before, during, and after EMI producing activities, allowing the ICD/VAD to function at optimal settings. This ability to suspend arrhythmia detection intermittently is unlike It differs from currently available magnets that once positioned over the ICD/VAD, triggering the asynchronous mode, the ICD/VAD cannot react to an arrhythmia detection unless the currently available magnet (all are permanent magnets) is physically removed from patient's chest.

These differences and other features of the present disclosure will become more fully apparent from the following description, or may be learned by the practice of the invention as set forth hereinafter.

### BRIEF DESCRIPTION OF THE SEVERAL VIEWS OF THE DRAWINGS

In order that the manner in which the above-recited and other features and advantages are obtained by the exemplary embodiments of this disclosure will be readily understood, a more particular description of the exemplary embodiments briefly described above will be rendered by reference to specific exemplary embodiments which are illustrated in the appended drawings. Understanding that these drawings depict only typical exemplary embodiments and therefore are not to be considered limiting in scope, the exemplary embodiments will be described and explained with additional specificity and detail through the use of the accompanying drawings in which:

FIG. 1 is a side elevation view of a patient lying supine showing an exemplary embodiment of an electromagnetic system with an exemplary electromagnet pad in relation to an MID, wherein a controller and power components are shown schematically;

FIG. 2 is a schematic view of an exemplary electromagnetic system showing the interior of an exemplary electromagnetic pad with a portion cut away to reveal the low-profile electromagnet and the cushioning gel;

FIG. 3 is a side elevation view of a patient lying supine showing another exemplary electromagnetic system with an exemplary electromagnet pad in relation to an MID, wherein a controller and power components of an electrocautery system are shown schematically;

FIG. 4 is a schematic view of an alternative, exemplary embodiment of the electromagnetic system showing the connection to an electrocautery system and the interior of an electromagnetic pad with a portion cut away to reveal an exemplary low-profile electromagnet; and

FIG. 5 is a schematic view of another alternative, exemplary embodiment of an electromagnetic system showing an alternative connection to a controller and an electrocautery system while showing the interior of an exemplary electromagnetic pad with a portion cut away to reveal an exemplary low-profile electromagnet.

### REFERENCE NUMBERS

| | |
|---|---|
| ICD/VAD defibrillator | electromagnet pad 12 |
| electromagnet system 10 | |
| controller 14 | power source 16 |
| patient 18 | medically implanted device (MID) 20 |
| wire 22 | wire 24 |
| electromagnet 26 | thin wafer 28 |
| conductive wire 30 | obverse panel 32 |
| reverse panel 34 | gel 36 |
| LED light indicator 38 | ON/OFF switch 40 |
| external battery pack 42 | external electrocautery generator 44 |
| grounding pad 46 | foot pedal 48 |
| electrocautery wand or pen 50 | signal wire 52 |
| delay circuitry 53 | electrocautery devices 54 |
| OFF/ON button 55 | dial 58 |

### DETAILED DESCRIPTION OF THE INVENTION

The exemplary embodiments of the present disclosure will be best understood by reference to the drawings, wherein like parts are designated by like numerals throughout. It will be readily understood that the components of the exemplary embodiments, as generally described and illustrated in the figures herein, could be arranged and designed in a wide variety of different configurations. Thus, the following more detailed description of the exemplary embodiments, as represented in the figures, is not intended to limit the scope of the invention, as claimed, but is merely representative of exemplary embodiments of the invention.

The word “exemplary” is used exclusively herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

The ICD/VAD defibrillator electromagnet system 10 has been developed in response to the deficiencies of clinical magnets currently available on the market, and in particular, in response to the problems of improper positioning of clinical magnets, movement of the clinical magnet on a patient's chest once positioned, draped, anesthetized, or placed in X ray/CT/MRI; as well as, the pressure wound potential from large and heavy clinical magnets and external forces from positioning a patient prone/lateral or for prolonged surgical duration.

The ICD/VAD defibrillator electromagnet system 10, as shown in FIG. 1, comprises an electromagnet pad 12, a controller 14 and a power source 16. As depicted, the electromagnet pad 12 is positioned on the skin surface of the chest of a patient 18 directly above a subsurface implanted MID 20, such as an ICD or a VAD. The electromagnet pad 12 is connected to the controller 14 via wire 22, and the controller 14 is connected to the power source 16 via wire 24. Wires 22, 24 carry electrical current from the power source 16, through the controller 14, to the electromagnet pad 12.

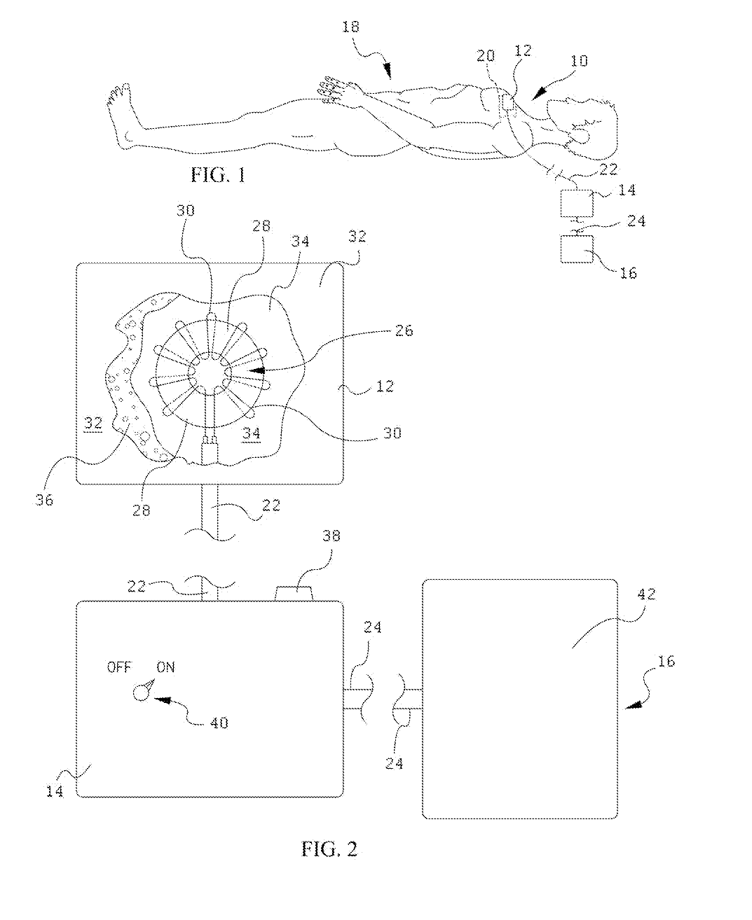

As indicated above, it is important that the exemplary electromagnet pad 12 is positioned and adhered to the chest of the patient 18 directly above the MID 20 in a manner designated by the manufacturer of the MID 20. The exemplary electromagnet (see FIG. 2) within the electromagnet pad 12 is made to be placed directly over a patient's ICD/VAD. The electromagnet pad 12 has an adhesive backing (not shown) that enables proper, temporary adhesion of the electromagnet to the skin despite difficult patient positioning (lateral/prone), body size, or length of procedure and provides protection from use of either a conductive or radiated EMI source. Such adhered positioning assures that the electromagnet within the electromagnet pad 12 can suspend arrhythmia detection of the MID 20 intermittently.

Turning now to FIG. 2, the exemplary electromagnet 26 comprises a thin wafer 28 (comprised of iron or a ferrous alloy or any other suitable metal that will become magnetic) that is wrapped in conductive wire 30 (such as copper or other conductive metallic wire) windings. Positive and negative poles are established so that conductive wire 30 carries an electrical current to activate the electromagnet 26. The number of times that the conductive wire 30 is wrapped (wound) around the thin wafer 28 will determine the strength of the electromagnet 26 for a given electrical current. As a result, electromagnets 26 may be made having various different strengths to match with the requirements for various MIDs 20. Of course, an adjustable electromagnet 26 may have a predetermined number of wrappings of the conductive wire 30 (as a standard) and the strength of the electromagnet 26 may be adjustable by adjusting the electrical current applied to the conductive wire 30. For purposes of this disclosure, the number of wrappings of the conductive wire 30 depicted in FIGS. 2, 4, and 5 have been limited so not to obscure the underlying thin wafer 28. Therefore, the wrappings depicted should not be construed to designate the number of wrappings that are required for the embodiments to operate as intended.

The electromagnet 26 is disposed inside the electromagnetic pad 12 which comprises an obverse panel 32 and reverse panel 34 that presents the electromagnet 26 in a low-profile configuration to significantly reduce the likelihood of pressure wounds and the dislodging of the electromagnet 26 from its intended position.

In an exemplary embodiment, the reverse panel 34 has an adhesive backing (not shown) to secure temporarily the electromagnetic pad 12 to the patient 18. Either or both of the obverse panel 32 and reverse panel 34 may have a non-conductive gel 36 within the panel 32, 34 to insulate the patient 18 from electrical shock while not affecting the magnetic field created by the electromagnet 26.

As shown in FIG. 2, the controller 14 may have an LED light indicator 38 that lights when the electromagnet 26 is activated. In some embodiments, the controller 14 may also have an ON/OFF feature such as an ON/OFF switch 40 that the medical provider may operate to turn the electromagnet 26 on or off at discretion. The controller 14, in some embodiments, may operate additional devices other than the electromagnet 26, as will be described below. It should be understood that a skilled artisan will know how to configure the circuitry within the controller 14 to turn the electromagnet 26 on and off, illuminate the LED light indicator 38 or other indicators (such as other lights or audible alerts), and operate other devices such as, for example, an electrocautery pen or wand.

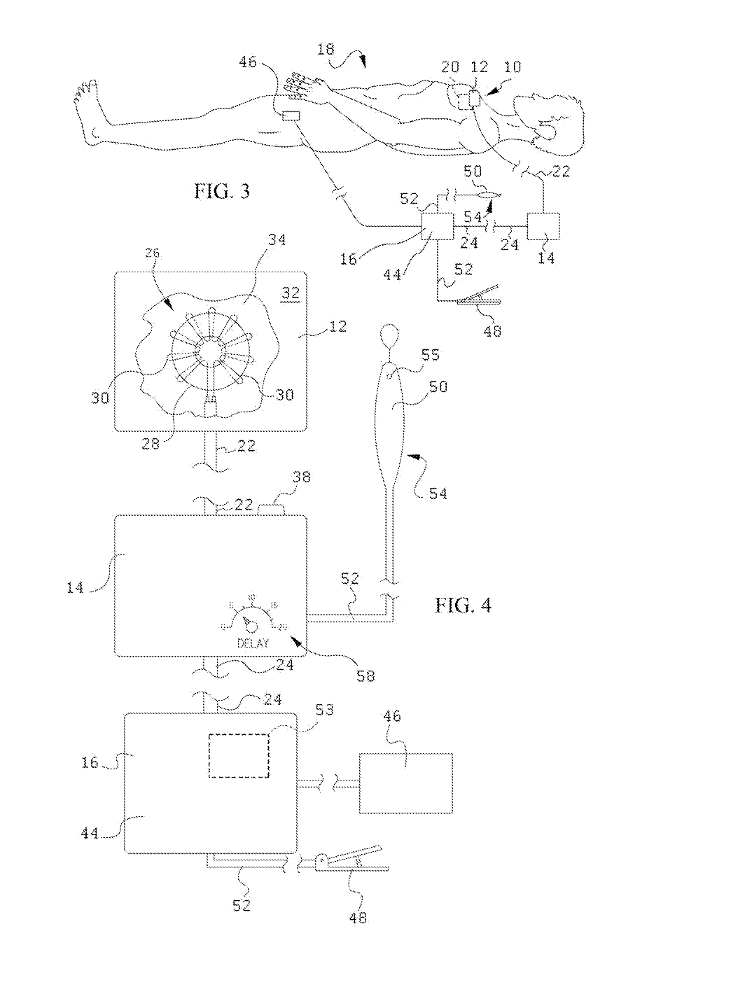

The power source 16 depicted in FIG. 1 may be any suitable power source 16 such as, for example, an external battery pack 42 (FIG. 2), an AC power source and inverter (not shown, but contemplated), or an external electrocautery generator source (see FIGS. 3-5). As the strength of an electromagnet 26, having a given number of windings of the conductive wire 30, can be increased or decreased by increasing or decreasing the power to the electromagnet 26, it is contemplated that the power source 16 may have the capability to supply power adjustability to adjust the strength of the electromagnet 16 in some embodiments where the ability to adjust the strength of the electromagnet 26 would be advantageous.

When the electromagnet 26 is active, thereby creating a magnetic field, it causes the suspension of arrhythmia detection by the MID 20 so that the presence of EMI will not be misinterpreted as an arrhythmia event. The medical provider can turn the power to the electromagnet 26 off and on in any of a number of ways that are described herein so that the operation of the MID 20 is suspended during the likely presence of EMI.

FIGS. 3-5 illustrate two alternative, exemplary embodiments of the ICDS/VAD defibrillator electromagnet system 10. In FIGS. 3 and 4, the ICDS/VAD defibrillator electromagnet system 10 comprises an electromagnet pad 12, a controller 14 and a power source 16. As depicted, the electromagnet pad 12 is positioned on the skin surface of the chest of a patient 18 directly above a subsurface implanted MID 20, such as an ICD or a VAD. It should be understood that the electromagnet pad 12 is described as working to suspend the operation of MIDs such as ICDs and VADs, but the electromagnet pad 12 could be used to activate/deactivate any implanted or subcutaneous device that responds to the presence of a magnetic field by initiating/suspending operation until the magnetic field is removed. The electromagnet pad 12 is connected to the controller 14 via wire 22, and the controller 14 is connected to the power source 16 via wire 24. Wires 22, 24 carry electrical current from the power source 16, through the controller 14, to the electromagnet pad 12.

Again, it is important that the electromagnet pad 12 is positioned and adhered to the chest of the patient 18 directly above the MID 20 in a manner designated by the manufacturer of the MID 20. The electromagnet 26 within the electromagnet pad 12 is made to be placed directly over a patient's ICD/VAD. The electromagnet pad 12 has an adhesive backing (not shown) that enables proper, temporary adhesion of the electromagnet 26 proximate to the skin despite difficult patient positioning (lateral/prone), body size, or length of procedure and provides protection from use of either a conductive or radiated EMI source. Such positioning assures that the electromagnet 26 within the electromagnet pad 12 can suspend arrhythmia detection of the MID 20 intermittently.

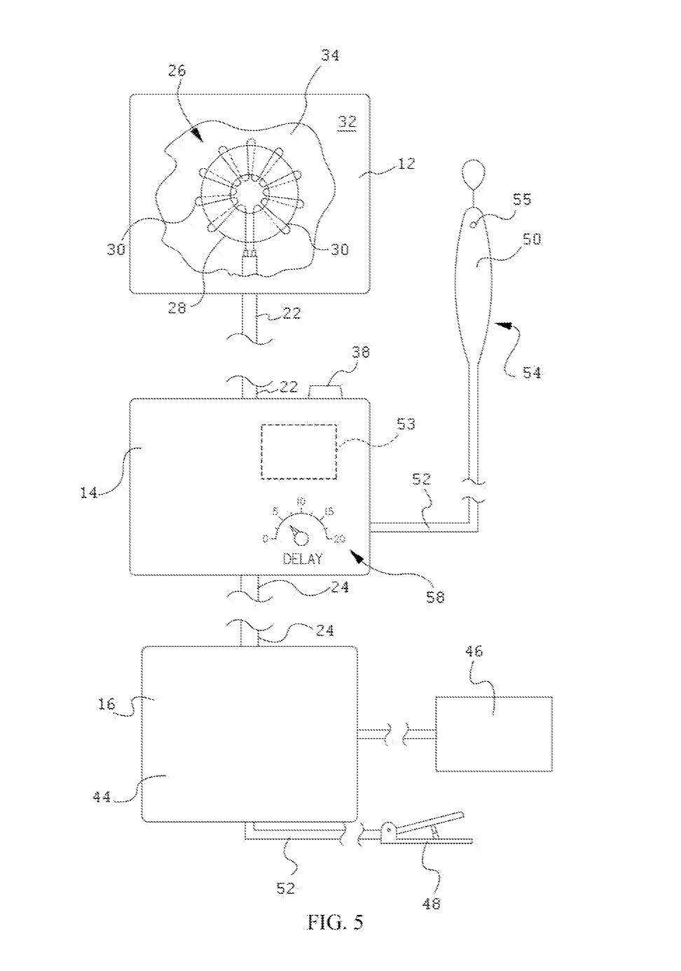

As described above, the exemplary electromagnet 26 comprises a thin wafer 28 that is wrapped in conductive wire 30. The thin wafer 28 may have any suitable shape such as disc, a ring or doughnut, a horseshoe, a rectangle or bar, or any other suitable shape that may deliver the desired magnetic field. Positive and negative poles are established so that conductive wire 30 carries an electrical current to activate the electromagnet 26. The electromagnet 26 is disposed inside the electromagnetic pad 12 which comprises an obverse panel 32 and reverse panel 34 that presents the electromagnet 26 in a low-profile configuration to significantly reduce the likelihood of pressure wounds and the dislodging of the electromagnet 26 from its intended position. In an exemplary embodiment, the reverse panel 34 has an adhesive backing (not shown) to secure temporarily the electromagnetic pad 12 to the patient 18. Either or both of the obverse panel 32 and reverse panel 34 may have a non-conductive gel 36 (not shown in FIG. 4 or 5) within the panel 32, 34 to insulate the patient 18 from electrical shock while not affecting the magnetic field created by the electromagnet 26.

The controller 14 may have LED light indicators 38 that light when the electromagnet 26 is activated and/or to signal various conditions existing in the system. Additional LED light indicators 38 also may be used to serve as visual alerts of the existence of various conditions such as an intensity threshold, the operation of an additional device, or any other desired alert. In the exemplary embodiments shown in FIGS. 3 and 4, the controller 14 may not have an ON/OFF switch 40 that the medical provider operates to turn the electromagnet 26 on or off at discretion. Rather, the operation of the electromagnet 26 is coincident with the power being provided by an external electrocautery generator 44 (e.g., a Bovie generator or an APC/Erby generator). The power source 16 may be any suitable power source 16 such as, for example, an external battery pack 42 (FIG. 2), an AC power source and inverter (not shown, but contemplated), or an external electrocautery generator 44 (see FIGS. 3-5).

FIGS. 3 and 4 show exemplary embodiments where the external electrocautery generator 44 controls the delivery of electrical current to the electromagnet 26 and determines when arrhythmia detection of the MID 20 is suspended. Controller 14 is connected to the external electrocautery generator 44 via wire 24. Of course, wire 24 may have an adaptor (not shown) that plugs into the external electrocautery generator 44 in a similar fashion as do other attachments. Those skilled in the art will understand what types of adaptors will serve to provide power from the external electrocautery generator 44 to the controller 14 for delivery to the electromagnet 26.

As shown, the external electrocautery generator 44 serves as the power source 16 and may control and/or provide power to several attachments. One attachment may be a grounding pad 46 that is secured to the patient 18 to provide an electrical ground for the system. Another attachment, for example, may be a foot pedal 48 that signals the power source 16 to supply power to an electrocautery wand or pen 50 which is another attachment. When the foot pedal 48 is depressed by the medical provider, a signal is sent through signal wire 52 to the power source 16. The power source 16 understands such signal as an instruction to provide power to the electrocautery wand or pen 50. In some embodiments, the power source 16 also understands such signal as an instruction to provide power to the controller 14 and the electromagnet 26. Hence, power is provided simultaneously to the electrocautery wand or pen 50, the controller 14, and the electromagnet 26. In this manner, the medical provider can turn the electromagnet 26 on and off at discretion by depressing and releasing the foot pedal 48.

In other exemplary embodiments, delay circuitry 53 is provided that delays the supply of power to the electrocautery wand or pen 50 until a short while after power is supplied to the electromagnet 26 so that arrhythmia detection of the MID 20 is suspended shortly before the electrocautery wand or pen 50 becomes active. The delay may be only a few seconds (for example, 5-10 seconds) or may be longer or shorter as desired. In fact, the delay may be adjustable within a range by moving a dial (see FIGS. 4 and 5 for example) supplied for that purpose. Additionally, the delay circuitry 53 may also cause a reverse delay that turns off the electrocautery wand or pen 50 shortly before the electromagnet 26 is turned off. As shown in FIGS. 3 and 4, the delay circuitry 53 (shown in schematic phantom lines) is internal to the external electrocautery generator 44.

The delay circuitry 53 may comprise a time relay resistor with a short delay (5-10 seconds, for example) that is provided so that the electromagnet 26 is activated shortly before the electrocautery device 54 and remains active until shortly after the electrocautery device 50 is deactivated. This resistor, once activated by external electrocautery generator 44, triggers the MID (ICD/VAD) 20, placing it in an asynchronous mode before the EMI source is activated and used, and maintains that asynchronous mode until after the EMI source is deactivated.

Some electrocautery devices 54 (such as the electrocautery pens 50 shown in FIGS. 4 and 5) have an OFF/ON button (or switch) 55 that turns the electrocautery device 54 off and on. Similar to the use of the foot pedal 48, when the OFF/ON button 55 is depressed or switch 55 is moved by the medical provider, a signal is sent through signal wire 52 to the external electrocautery generator 44. The external electrocautery generator 44 understands such signal as an instruction to provide power to the electrocautery wand or pen 50. In some embodiments, the external electrocautery generator 44 also understands such signal as an instruction to provide power to the controller 14 and the electromagnet 26. Hence, power is provided simultaneously to the electrocautery wand or pen 50, the controller 14, and the electromagnet 26. In this manner, the medical provider can turn the electromagnet 26 on and off at discretion by depressing and releasing the OFF/ON button 55 or by moving the switch 55.

Similarly, in some exemplary embodiments, delay circuitry 53 is provided that delays the supply of power to the electrocautery wand or pen 50 until a short while after power is supplied to the electromagnet 26 so that arrhythmia detection of the MID 20 is suspended shortly before the electrocautery wand or pen 50 becomes active. The delay may be only a few seconds (for example, 5-10 seconds) or may be longer or shorter as desired. In fact, the delay may be adjustable within a range by moving a dial 58 supplied for that purpose. Additionally, the delay circuitry may also cause a reverse delay that turns off the electrocautery wand or pen 50 shortly before the electromagnet 26 is turned off. As shown in FIG. 4, the delay circuitry 53 is internal to the external electrocautery generator 44. In this case, the dial 58 may be located on the controller 14. In other embodiments, the delay circuitry 53 may be located in the external electrocautery generator 44 and the dial 58 may be on the external electrocautery generator 44 (not shown) or on a separate box (not shown) that could be located for easy access by the operator.

Turning now to FIG. 5, the exemplary embodiment of the ICD/VAD defibrillator electromagnet system 10 shown utilizes controller 14 to cause the delay described above. Instead of having the delay circuitry 53 in the external electrocautery generator 44, such delay circuitry 53 is provided in the controller 14. In this manner, the external electrocautery generator 44 need not be modified to have such circuitry. Rather, the controller 14 with delay circuitry 53 may be connected to the external electrocautery generator 44 to retrofit the delay capability to the system without affecting the operation of the external electrocautery generator 44.

FIG. 5 shows an exemplary embodiment of the ICD/VAD defibrillator electromagnet system 10 where the controller 14 controls the delivery of electrical current to the electromagnet 26 and determines when arrhythmia detection of the MID 20 is suspended. Controller 14 is connected to the external electrocautery generator 44 via wire 24. Of course, wire 24 may have an adaptor (not shown, but known to those skilled in the art) that plugs into the external electrocautery generator 44 in a similar fashion as do other attachments. Such adaptors may have one or more male prongs that engage corresponding female receptacles to provide a coupling required for the operation of the system as desired. The controller 14 may also have a receptacle (not shown) similar to what is provided on the external electrocautery generator 44 for receiving an adaptor (such as a male jack) on signal wire 52 connected to the electrocautery wand or pen 50. Those skilled in the art will understand what types of adaptors will serve to provide power from the external electrocautery generator 44 to the controller 14 for delivery to the electromagnet 26 and to the electrocautery wand or pen 50.

As shown, the external electrocautery generator 44 may have a grounding pad 46 attachment that is to be secured to the patient 18 to provide an electrical ground for the system, and optionally a foot pedal 48 that signals the power source 16 to supply power to the controller 14 which in turn supplies power to the electromagnet 26 and the electrocautery wand or pen 50. When the foot pedal 48 is depressed by the medical provider, a signal is sent through signal wire 52 to the external electrocautery generator 44. The external electrocautery generator 44 understands such signal as an instruction to provide power to the electrocautery wand or pen 50 via the controller 14. Hence, power is provided to the controller 14 which activates the electromagnet 26. In this manner, the medical provider can turn the electromagnet 26 on and off at discretion by depressing and releasing the foot pedal 48.

In the exemplary embodiment shown, delay circuitry 53 is provided in the controller that delays the supply of power to the electrocautery wand or pen 50 until a short while after power is supplied to the electromagnet 26 so that arrhythmia detection of the MID 20 is suspended shortly before the electrocautery wand or pen 50 becomes active. The delay may be only a few seconds (for example, 5-10 seconds) or may be longer or shorter as desired. In fact, the delay may be adjustable within a range by moving a dial 58 supplied for that purpose. The dial 58 may be located on the controller 14 or on a separate box (not shown) that could be located for easy access by the operator.

Additionally, the delay circuitry 53 may also cause a reverse delay that turns off the electrocautery wand or pen 50 shortly before the electromagnet 26 is turned off. As shown in FIG. 5, the delay circuitry 53 is internal to the controller 14 and provides a retrofit delay capability to the overall system.

The delay circuitry 53 may comprise a time relay resistor with a short delay (5-10 seconds, for example) that is provided so that the electromagnet 26 is activated shortly before the electrocautery device 54 and remains active until shortly after the electrocautery device 54 is deactivated. This resistor, once activated by controller 14, triggers the ICD/VAD, placing it in an asynchronous mode before the EMI source is activated and used, and maintains that asynchronous mode until after the EMI source is deactivated.

Again, the controller may have an LED light indicator 38 that illuminates when the electromagnet 26 is active. This embodiment also allows the external electrocautery generator 44 to power the electromagnet 26 through the controller 14 and to serve as a grounding source as well.

The defibrillator electromagnet structure of the exemplary embodiments of the present invention may have the same or similar ferrous alloy design, but has a substantially slimmer profile design, less weight and has an adhesive pad to help with better positioning and adhesion of electromagnet compared to current market magnets. The ability to trigger the asynchronous mode through an external battery pack, external generator, or other power source allows for more effective management of patient's hemodynamic status and allows the ICD/VAD to function at optimal settings to benefit the patient.

The defibrillator electromagnet is a non-invasive ICD/VAD electromagnet that has a low profile and is disposed in a non-conductive adhesive gel pad to aide with correct positing over the ICD/VAD. Its slim profile also decreases the risk of pressure wounds due to prone/supine/lateral positioning or prolonged procedures. The electromagnet's ability to have arrhythmia detection suspended intermittently at medical provider discretion or in concert with an external generator activation by the medical provider before, during, and after EMI producing activities, allows the ICD/VAD to function at optimal settings. It differs from current magnets available that once positioned over the ICD/VAD, triggering the asynchronous mode, the ICD/VAD cannot react to an arrhythmia detection unless the permanent magnet is removed from patient's chest.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A defibrillator electromagnet structure for attachment to a patient and providing a magnetic field for communicating with a medically implanted device to activate or deactivate the medically implanted device when an operator activates a power source to supply power to the defibrillator electromagnet structure, the defibrillator electromagnet structure comprising:
a electromagnet comprising a thin wafer and conductive wire wrapped around the thin wafer, the electromagnet becoming active upon the receipt of power from the power source;
a cushioning pad encasing the electromagnet; and
a controller connected to the electromagnet and to the power source for supplying power to the electromagnet to activate the electromagnet.

2. A defibrillator electromagnet structure as in claim 1 wherein the cushion pad has a low-profile and an adhesive to facilitate attachment to the patient.

3. A defibrillator electromagnet structure as in claim 1 wherein the cushion pad has an obverse panel and a reverse panel, at least one of the obverse panel and the reverse panel containing a non-conductive gel.

4. A defibrillator electromagnet structure as in claim 1 further comprising an ON/OFF feature for the operator to activate and deactivate the electromagnet selectively at the operator's discretion.

5. A defibrillator electromagnet structure as in claim 4 wherein the ON/OFF feature is selected from the group consisting of an ON/OFF switch on the controller, an ON/OFF switch on the power source, an ON/OFF button on the controller, an ON/OFF button on the power source, a foot pedal, and a ON/OFF button on an electrocautery device.

6. A defibrillator electromagnet structure as in claim 1 wherein the strength of the electromagnet is adjustable by adjusting the amount of power supplied to the controller by the power source.

7. A defibrillator electromagnet structure as in claim 1 wherein the strength of the electromagnet is adjustable by adjusting the amount of power supplied to the electromagnet by the controller.

8. A defibrillator electromagnet structure as in claim 1 wherein the controller comprises an LED light indicator.

9. A defibrillator electromagnet structure as in claim 8 wherein the LED light indicator emits light when the electromagnet is active.

10. A defibrillator electromagnet structure as in claim 1 wherein the controller comprises delay circuitry that activates the electromagnet a predetermined amount of time before the patient will be exposed to EMI.

11. A defibrillator electromagnet structure as in claim 10 wherein the delay circuitry that deactivates the electromagnet a predetermined amount of time after the patient is no longer exposed to EMI.

12. A defibrillator electromagnet system for attachment to a patient and providing a magnetic field for communicating with a medically implanted device to activate or deactivate the medically implanted device when an operator activates a power source to supply power to the defibrillator electromagnet structure, the defibrillator electromagnet structure comprising:
a electromagnet comprising a thin wafer and conductive wire wrapped around the thin wafer, the electromagnet becoming active upon the receipt of power from the power source;
a cushioning pad encasing the electromagnet;
a controller connected to the electromagnet and to the power source for supplying power to the electromagnet to activate the electromagnet; and
a grounding pad connected to the controller for application to the patient to electrically ground the defibrillator electromagnet system.

13. A defibrillator electromagnet system as in claim 12 wherein the cushion pad has an obverse panel and a reverse panel, at least one of the obverse panel and the reverse panel containing a non-conductive gel.

14. A defibrillator electromagnet system as in claim 12 further comprising an ON/OFF feature for the operator to activate and deactivate the electromagnet selectively at the operator's discretion.

15. A defibrillator electromagnet system as in claim 12 wherein the strength of the electromagnet is adjustable by adjusting the amount of power supplied to the controller by the power source.

16. A defibrillator electromagnet system as in claim 12 wherein the strength of the electromagnet is adjustable by adjusting the amount of power supplied to the electromagnet by the controller.

17. A defibrillator electromagnet system as in claim 12 further comprising delay circuitry for activating the electromagnet a first predetermined amount of time before the patient will be exposed to EMI and deactivating the electromagnet a second predetermined amount of time after the patient is no longer exposed to EMI.

18. A method for suspending arrhythmia detection capability of a medically implanted device implanted within a patient, comprising the steps of:
positioning an electromagnet in a desired location over the location of the medically implanted device;
adhering the electromagnet to the patient at the desired location;
supplying power to the electromagnet to activate the electromagnet to create a magnetic field; and
suspending arrhythmia detection capability of the medically implanted device by applying the magnetic field to the device.

19. A method for suspending arrhythmia detection capability of a medically implanted device as in claim 18 wherein the step of supplying power comprises supplying power to the electromagnet before supplying power to an electromagnetic interference-producing device proximate to the patient.

20. A method for suspending arrhythmia detection capability of a medically implanted device as in claim 19 further comprising the step of discontinuing power to the electromagnet to deactivate the electromagnet to remove the magnetic field after power to the electromagnetic interference-producing device proximate to the patient is discontinued.
